(19) United States
(12) Patent Application Publication
(10) Pub. No.: US 2023/0310785 A1
(43) Pub. Date: Oct. 5, 2023

(54) RESUSCITATION MASK HAVING CUFF ADHESIVE SEAL WITH REMOVABLE TAPE BACKING, KIT WITH REPLACEMENT MASK SEALS AND METHOD OF USE THEREOF

(71) Applicant: CARDIAC RESPONSE YYC LTD., Calgary (CA)

(72) Inventor: Vicher Angelou Tenebro, Calgary (CA)

(21) Appl. No.: 18/295,390

(22) Filed: Apr. 4, 2023

(30) Foreign Application Priority Data
Apr. 5, 2022 (CA) 3154166

(51) Int. Cl. A61M 16/06

(52) U.S. Cl. CPC A61M 16/0688; A61M 16/0616

(57) ABSTRACT

A mask kit includes a resuscitation mask with an adhesive tape around the soft cuff. The adhesive tape layer is a dual-sided tape affixed to the bottom cuff and having a removable tape backing affixed thereto. The kit further includes replacement tape seals, each replacement tape seal having a two-sided adhesive tape with removable tape backing on both sides. Usage of the kit involves removing the tape backing on the mask cuff, affixing the cuff with adhesive layer to the patient’s face, and performing CPR or another medical procedure on a patient using the mask. After usage, the mask is cleaned and one removable tape backing of a replacement tape is seal is removed. The adhesive layer of the replacement tape seal is affixed to cover the used adhesive layer on the cuff of the mask. The mask is then returned to the kit ready to use a next time.

### CROSS-REFERENCE TO RELATED APPLICATIONS

This application claims the benefit of priority of Canadian Patent Application No. 3,154,166 filed Apr. 05, 2022, which is incorporated herein by reference.

### BACKGROUND OF THE INVENTION

### Field of the Invention

The invention pertains generally to face masks utilized when providing emergency cardiopulmonary resuscitation (CPR). More specifically, the invention relates to removable tape backings, resuscitation mask kits and method thereof for facilitating repeatedly securing a CPR mask to different patients for repeated use.

### Description of the Related Art

A cardiopulmonary resuscitation (CPR) mask, also referred to as a resuscitation mask, pocket mask or pocket face mask, is a device used by a first responder to safely perform rescue breathing during a cardiac arrest or respiratory arrest of a patient.

FIG. 1 illustrates a top and side perspective view of a conventional CPR mask. The conventional mask has a cone-shape canopy with a soft cuff extending around its edge, which is applied against the skin of the patient around the nose and mouth. A port opens into the interior of the canopy so that air from the breaths of a first responder can be supplied to the patient’s nose and mouth.

Usually, these face masks are held against the patient’s face manually or by means of a strap extending around the patient’s head. However, the strap is ineffective and often not utilized and it can also be difficult to achieve an effective seal without the first responder applying manual pressure and this is a disadvantage because it occupies the time and attention of the first responder. There is only a certain amount of time such as ten seconds to gives two breaths before the first responder must switch to doing chest compressions. Wasted time during the breathing portion of the CPR cycle is best to be avoided.

U.S. Pat. No. 8,720,438 entitled “BREATHING MASK WITH STICKY EDGE” and U.S. Pat. No. 6,851,429 entitled “FACE MASKS” disclose face masks secured to a patient’s face by means of an adhesives, sticky gel or putty, and/or adhesive gaskets. Although these adhesive attachments have certain advantages, they are not well-suited to situations where the same mask needs to later be reused on different patients.

### BRIEF SUMMARY OF THE INVENTION

According to an exemplary embodiment of the invention there is disclosed a resuscitation mask with an adhesive tape around the soft cuff of the mask.

According to an exemplary embodiment of the invention there is disclosed a kit including a resuscitation mask with adhesive tape layer being a dual sided tape with a first affixed to the bottom cuff and a second side having a removable tape backing affixed thereto. The kit further includes a plurality of replacement tape seals, each replacement tape seal having a two-sided adhesive tape with removable tape backing on both sides.

According to an exemplary embodiment of the invention there is disclosed a method of usage of the kit involving removing the tape backing on the bottom of the mask cuff, affixing the adhesive layer on the cuff to the patient’s face when putting the mask on the patient, and performing CPR or another medical procedure on a patient using the mask. After usage, the mask is cleaned and the removable tape backing on one side of a replacement tape is seal is removed. The adhesive layer of the replacement tape seal is then affixed to cover the used adhesive layer on the cuff of the mask. The mask is then returned to the kit ready to use a next time.

According to an exemplary embodiment of the invention there is disclosed a refill package including one or more replacement tape seals. In some embodiments, each replacement tape seal includes a dual-sided tape layer with removable tape backing layers on both sides. In some embodiments, each replacement tape seal in the refill package includes a respective one-way valve.

These and other advantages and embodiments of the present invention will no doubt become apparent to those of ordinary skill in the art after reading the following detailed description of preferred embodiments illustrated in the various figures and drawings.

### BRIEF DESCRIPTION OF THE DRAWINGS

The invention will be described in greater detail with reference to the accompanying drawings which represent preferred embodiments thereof:

FIG. 1 illustrates a top and side perspective view of a conventional CPR mask according to the prior art.

FIG. 2 shows an exploded side-view of a CPR mask according to an exemplary embodiment of the present invention.

FIG. 3 shows a side-view of the assembled CPR mask of FIG. 2 ready for storage and future use according to an exemplary embodiment.

FIG. 4 illustrates a bottom plan view of the assembled CPR mask of FIG. 3 with tape backing fully covering the adhesive tape layer according to an exemplary embodiment.

FIG. 5 illustrates a bottom plan view of the assembled CPR mask of FIG. 3 after a user has started to remove the removable tape backing to thereby expose the sticky underside of the adhesive tape layer.

FIG. 6 illustrates a bottom plan view of the assembled CPR mask of FIG. 4 after a user has completely removed the removable tape backing.

FIG. 7 illustrates an exploded side view of three layers of a replacement tape seal according to an exemplary embodiment.

FIG. 8 illustrates a top plan view of a replacement tape seal with the top removable tape backing fully in place.

FIG. 9 illustrates a top plan view of the replacement tape seal of FIG. 8 after a user has started to peel back the top removable tape backing to expose the top layer of the two-side adhesive.

FIG. 10 illustrates an exploded side view illustrating a process of resetting a used CPR mask back to a state ready for storage and future usage according to an exemplary embodiment.

FIG. 11 illustrates a resuscitation mask kit including a fully assembled CPR mask as illustrated in FIG. 4 and described above.

### DETAILED DESCRIPTION

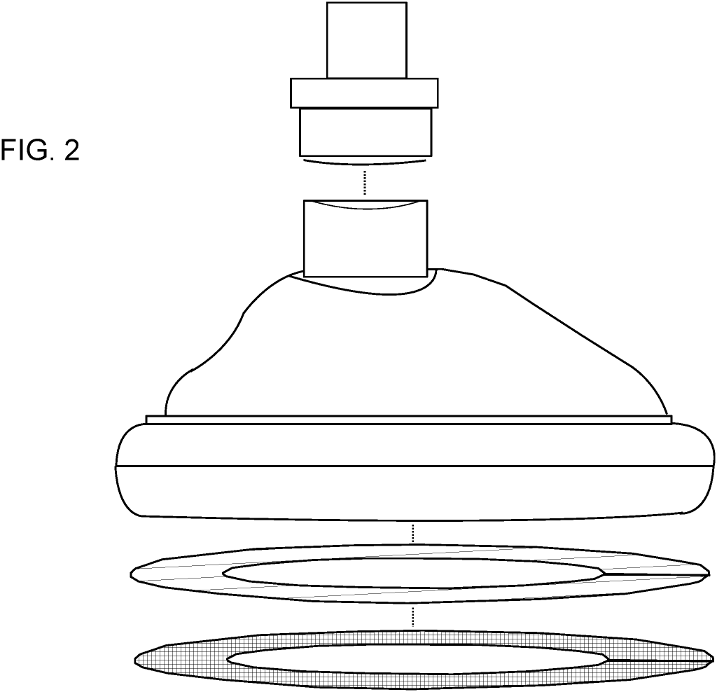

FIG. 2 shows an exploded side-view of a CPR mask according to an exemplary embodiment of the present invention. In this embodiment, the mask includes a one-way valve that affixes to a port on the top of the mask. The mark has a cone-shape canopy with a soft cuff extending around its edge and the underside of the soft cuff includes a two-sided adhesive tape with a removable tape backing. A first side of the adhesive tape is affixed to the underside of the soft cuff of the mask and the removable tape backing covers the second side of the adhesive tape backing until the mask is needed to a be used.

In some embodiments, the adhesive tape is a clear transparent tape suitable for all fabric types and safe for all skin types. Such double-sided tape is known, for example, in the women’s fashion industry and is sold as a double sided tape for clothing and body attachments. Other types of adhesive tapes safe for human skin may be used as desired in different embodiments.

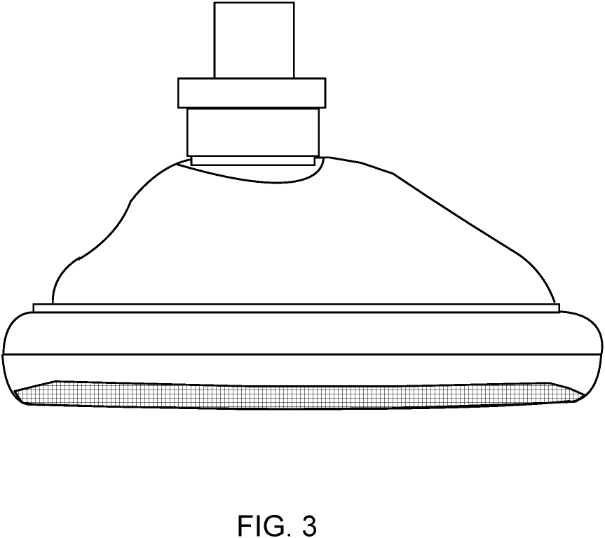

FIG. 3 shows a side-view of the assembled CPR mask of FIG. 2 ready for storage and future use according to an exemplary embodiment. The two-sided adhesive tape layer is sandwiched between the soft cuff of the mask and the removable tape backing. The underside of the tape backing is itself not sticky and therefore the bottom of the mask is not sticky and the mask is the assembled state of FIG. 2 can be stored in any suitable container such as within a pocket-sized case or bag.

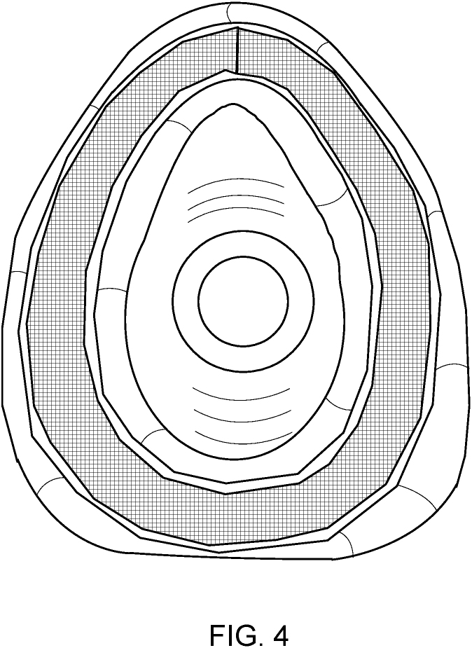

FIG. 4 illustrates a bottom plan view of the assembled CPR mask of FIG. 3 with tape backing fully covering the adhesive tape layer according to an exemplary embodiment. As shown, both the two-sided adhesive tape layer and the removable tape backing form an oval shape to match the shape and follow the oval perimeter of the bottom of soft cuff of the mask. One or more cut lines on the removable tape backing ends are present to facilitate a first responder or other user to grab a corner of the tape backing for removal prior to affixing the mask to a patient’s face.

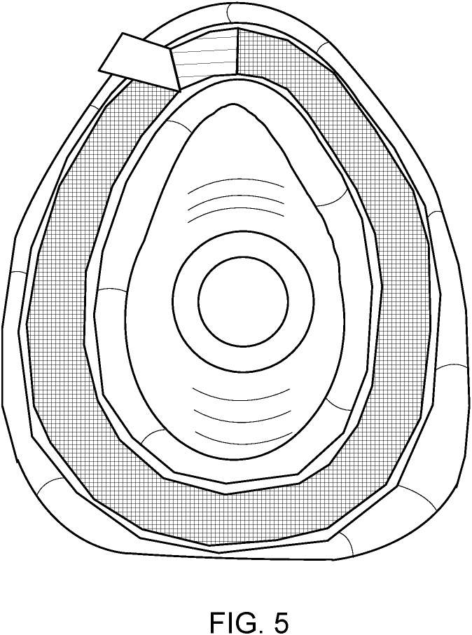

FIG. 5 illustrates a bottom plan view of the assembled CPR mask of FIG. 3 after a user has started to remove the removable tape backing to thereby expose the sticky underside of the adhesive tape layer. When the mask is to be used on a patient, the first responder or other user peels back the removable tape backing to separate it from the adhesive tape layer such that the adhesive tape layer is exposed and ready to affix to a patient’s face.

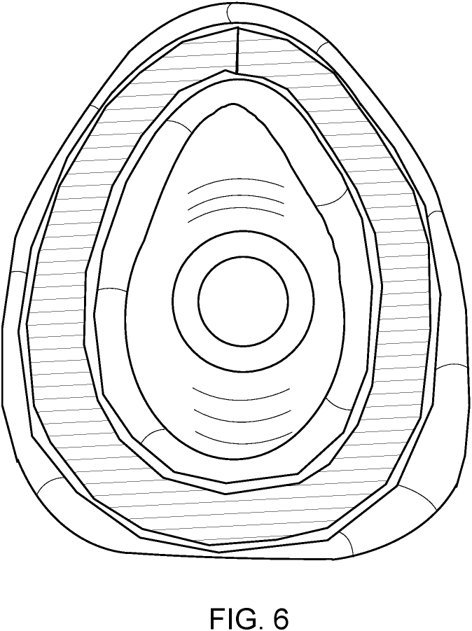

FIG. 6 illustrates a bottom plan view of the assembled CPR mask of FIG. 4 after a user has completely removed the removable tape backing. In this configuration, the CPR mask is ready for applying to a patient’s face. The underside of the adhesive tape layer is now fully exposed and ready to be affixed to a patient’s face.

An example of a method of utilizing the mask in an emergency situation is as follows. A user such as a first responder carries the CPR mask in the fully assembled state of FIGS. 3 and 4 in a storage container. When a victim or other patient needs emergency CPR, the user removes the mask from the storage containers, peels off the removable tape backing, and presses the soft cuff on the underside of the mask to the patient’s face thereby pressing the exposed side of the two-sided adhesive tape against the patient’s face. This action affixes the mask to the patient’s face and CPR can be performed in the usual manner with an effective seal assisted by the adhesive tape.

After the CPR is finished and the mask is finished being utilized on this patient, the first responder user peels the mask away from the patient’s face. At this point, the one-way valve may be removed and discarded, and the remaining mask body may be wiped down with disinfectant or otherwise cleaned and dried for reuse. A new one-way valve is installed and a replacement tape seal is applied to the mask cuff prior to storage.

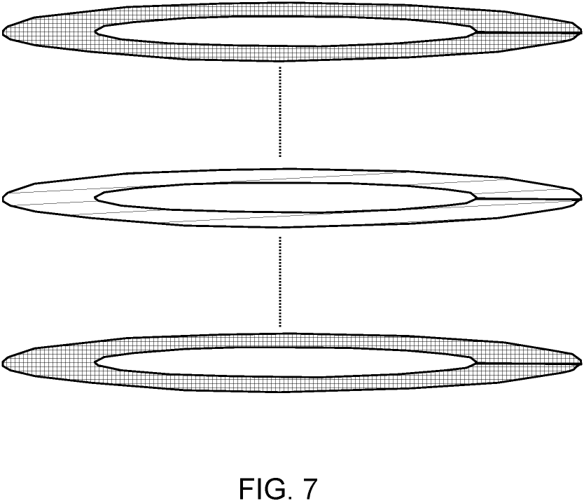

FIG. 7 illustrates an exploded side view of three layers of a replacement tape seal according to an exemplary embodiment. Each replacement tape seal is an oval shaped ring having three layers: a top removable tape backing, a two-sided adhesive tape, and a bottom removable tape backing. The two-sided adhesive tape layer is sandwiched between the top and bottom removable tape backing layers. A plurality of replacement tape seals may be included within the storage case along with the mask for resetting the mask back to a ready-for-storage-and-future-use state after each use.

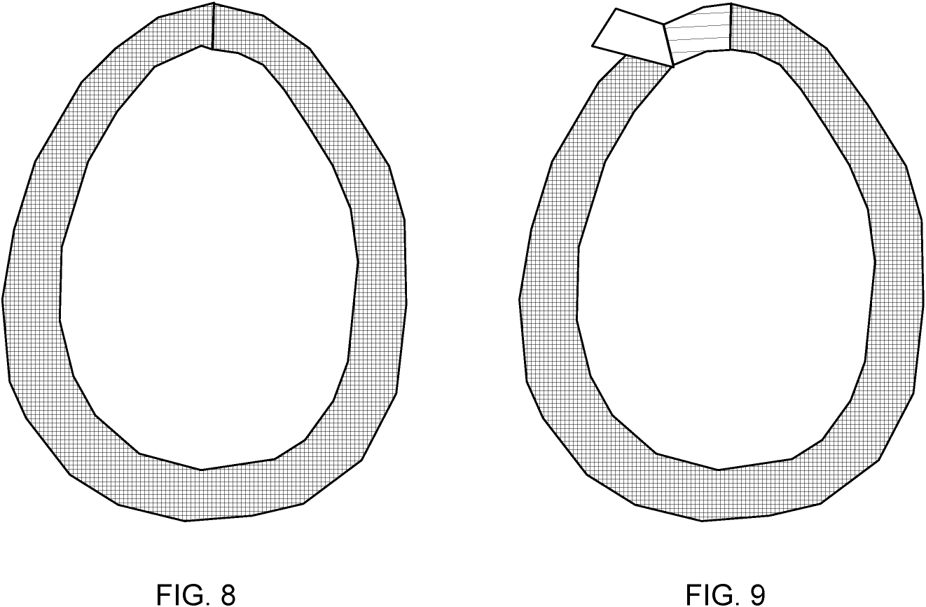

FIG. 8 illustrates a top plan view of a replacement tape seal with the top removable tape backing fully in place. FIG. 9 illustrates a top plan view of the replacement tape seal of FIG. 8 after a user has started to peel back the top removable tape backing to expose the top layer of the two-side adhesive.

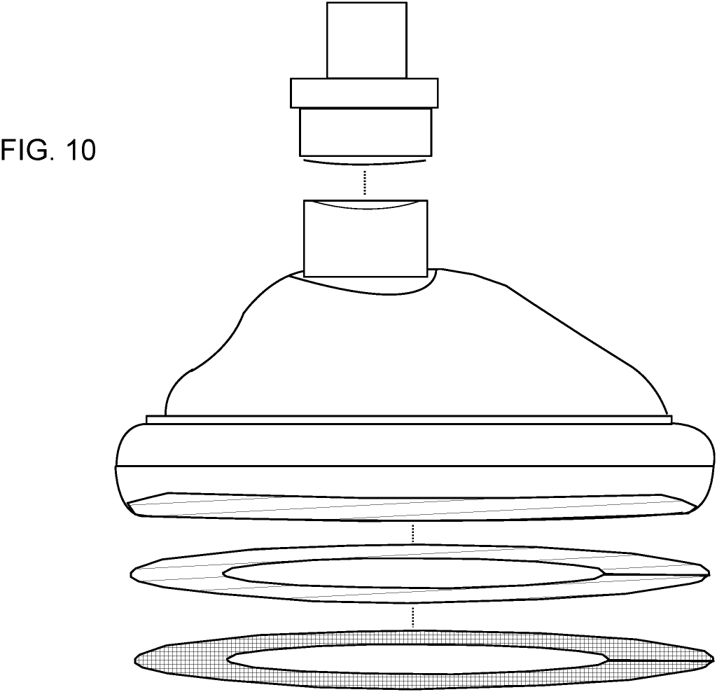

FIG. 10 illustrates an exploded side view illustrating a process of resetting a used CPR mask back to a state ready for storage and future usage according to an exemplary embodiment. A new one-way valve is installed to the air port on the top of the mask canopy. In preferred embodiments, the one-way valve is a single-use component that is discarded rather than being reused for sanitary reasons. Along the soft cuff on the underside of the mask, the replacement tape seal having its top removable tape backing removed is affixed against the adhesive tape layer that is already on the soft cuff from the previous use. In this way, the upward-facing side of the adhesive layer of the replacement mask seal joins with and fully covers the bottom-facing side of the existing adhesive layer on the mask. In other words, the new replacement tape seal covers the old one. The new replacement seal still includes the bottom removable tape layer and thus the configuration of the CPR mask after the layers of FIG. 10 have been assembled returns back to a state similar to as illustrated above in FIG. 4. The mask in this assembled state is ready to be placed back into the storage container for later usage.

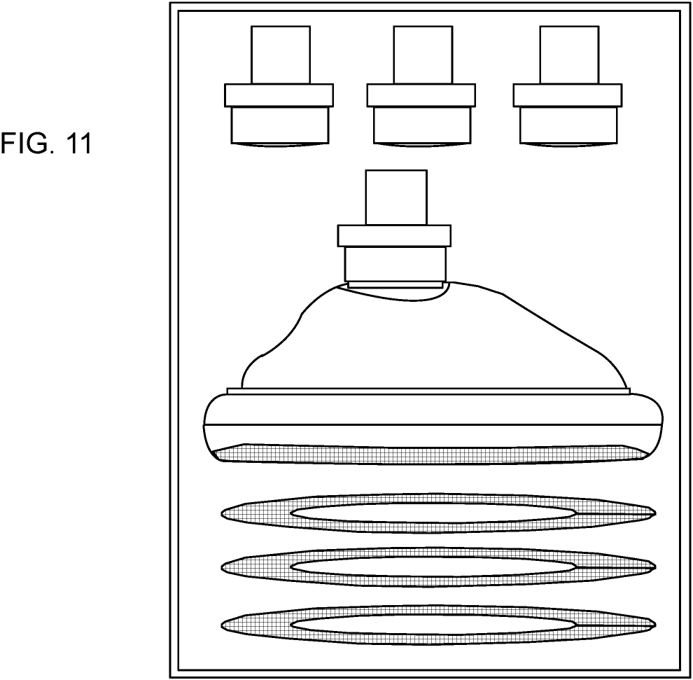

FIG. 11 illustrates a resuscitation mask kit including a fully assembled CPR mask as illustrated in FIG. 4 and described above. The kit further includes a plurality of three replacement one-way valves and a plurality of three replacement tape seals. Each of the tape seals include the three layers as illustrated in FIG. 7. The items of the kit may be stored in a case such as a pocket case or other container. The assembled CPR mask is ready to use by the quick removal of the bottom tape back backing as described above. After use, the mask is cleaned, the one-way valve is replaced using one of the replacement valves in the kit. Further, one side of removable tape backing of a replacement tape seal from the kit is removed to thereby expose the adhesive tape layer, and the exposed adhesive tape layer of the replacement tape seal is affixed to cover the previously used replacement adhesive tape layer on the cuff. The mask is now back to the state ready for storage and future usage.

In an exemplary embodiment, a mask kit includes a resuscitation mask with an adhesive tape around the soft bottom cuff of the mask. The adhesive tape layer is a dual sided tape with a first side affixed to the bottom cuff and a second side having a removable tape backing affixed thereto. The kit further includes a plurality of replacement tape seals, each replacement tape seal having a two-sided adhesive tape with removable tape backing on both sides. Usage of the kit involves removing the tape backing on the bottom of the mask cuff, affixing the adhesive layer on the cuff to the patient’s face when putting the mask on the patient, and performing CPR or another medical procedure on a patient using the mask. After usage, the mask is cleaned and the removable tape backing on one side of a replacement tape is seal is removed. The adhesive layer of the replacement tape seal is affixed to cover the used adhesive layer on the cuff of the mask. The mask is then returned to the kit ready to use a next time.

A benefit of some embodiments of the invention is that the mask kit facilitates reusing the mask on a new patient in the future. For instance, an individual may carry the kit in their vehicle and then perform CPR on a victim of a car accident, drowning or other emergency situation. After usage, the individual may affix the replacement seal back on the bottom of the mask and return the mask to the kit container. Thereafter, upon encountering a subsequent emergency situation, the mask is ready for use with the quick peeling back of the bottom removable tape backing on the replacement seal just like the initial usage.

The plurality of replacement tape seals in the kit further facilitate using the same mask for training purposes and for annual re-certification examinations commonly required for certain types of first responder and medical personnel.

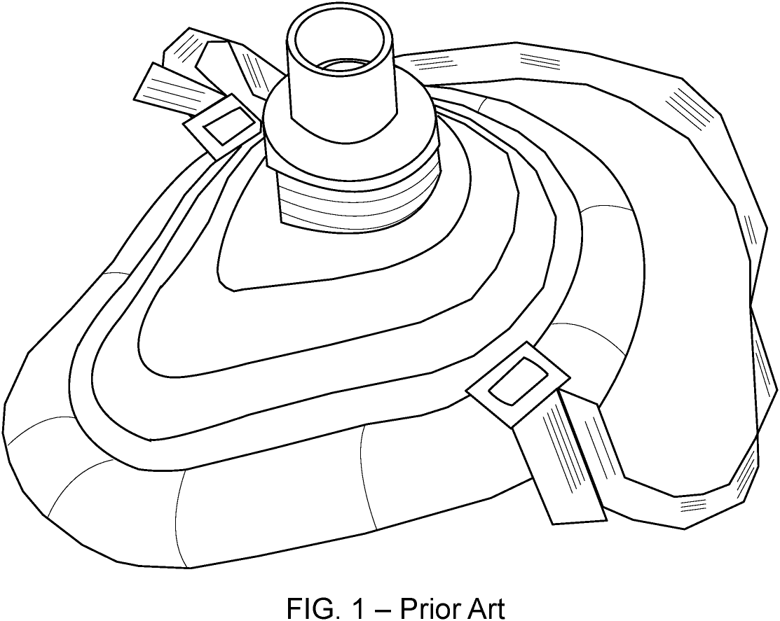

Although the invention has been described in connection with preferred embodiments, it should be understood that various modifications, additions and alterations may be made to the invention by one skilled in the art without departing from the spirit and scope of the invention. For example, the one-way valve and the mask body with soft cuff may in some embodiments be conventional masks such as shown in FIG. 1 including strap attachments and/or strap.

The above-described steps of using the CPR mask may be performed by a user such as a first responder but may also be performed by other users such as clinicians or nurses and doctors, for example.

Although the above examples of have focused on CPR as the application with which the resuscitation mask is utilized, the same or similar masks having adhesive tape seals may also be used in similar manner in other applications. For example, rather than a CPR mask, the mask may be a bag valve mask (BVM) device for hospital setting usage.

The replacement tape seals may also be sold separately from the mask and mask kit. For example, a set of ten replacement tape seals may be sold as a refill package to restock an existing kit without needing to purchase a new mask or kit container. In some embodiments, the replacement tape seals are sold with a corresponding set of one-way valves. In one example embodiment, a refill package including ten replacement tape seals also includes a respective plurality of ten replacement one-way valves.

The above-described steps are not restricted to the exact order described, and, in other configurations, described steps may be omitted or other intermediate steps added. Functions of single parts may be separated into multiple parts, or the functions of multiple parts may be combined into a single unit. For example, rather than having replacement tape seals that are each an integral oval shape as illustrated above, a plurality of arced or straight replacement tape seal sections may be included in the kit. The user may apply a plurality of replacement tape seal sections over the existing adhesive tape on the bottom cuff of the mask to thereby cover the old tape seal and place the mask back into the ready-for-storage configuration of FIG. 4. This may be beneficial such that the replacement seal tape sections can be used with different sized and shaped masks, for example. All combinations and permutations of the above described features and embodiments may be utilized in conjunction with the invention.

What is claimed is:

1. An apparatus as shown or described herein.

2. A kit as shown or described herein.

3. A method as shown or described herein.
